(19) United States
(12) Patent Application Publication
(10) Pub. No.: US 2015/0105713 A1
(43) Pub. Date: Apr. 16, 2015

(54) ELECTRICAL MUSCLE STIMULATION (EMS)- Buffer pads

(71) Applicant: Rael Gilchrist, Hollywood, FL (US)

(72) Inventor: Rael Gilchrist, Hollywood, FL (US)

(21) Appl. No.: 14/509,485

(22) Filed: Oct. 8, 2014

Related U.S. Application Data
(60) Provisional application No. 61/889,400, filed on Oct. 10, 2013.

(51) Int. Cl. A61N 1/04

(52) U.S. Cl. CPC A61N 1/0428; A61N 1/0452; A61N 1/0456
USPC 604/20; 607/115

(57) ABSTRACT

An electrode cover for use with a medical electrode is formed of a main body having a substantially planer configuration with a distal surface constructed to mate with an electrode and a proximal surface constructed for placement on a user's skin, wherein the main body is constructed and arranged with thickness and porosity to deliver 95.0-99.9% of electrical energy transmitted from said medical electrode to the skin surface of a patient.

### INDEX TO RELATED APPLICATIONS

This application is a non-provisional of, and claims benefit to U.S. Provisional Patent Application Ser. No. 61/889,400 filed Oct. 10, 2013 the disclosure of which is incorporated herein by reference in its entirety.

### BACKGROUND OF THE INVENTION

Carbon rubber electrode pads are utilized to provide electrical contact with a subjects skin. The carbon pad is highly conductive and transmits electrical signals to the patient's skin and muscles. These carbon electrodes are utilized for Transcutaneous Electrical Nerve Stimulation (T.E.N.S), and Electrical Muscle Stimulation (E.M.S.). Transcutaneous Electrical Nerve Stimulation is useful, for example, in post-operative and chronic pain control, while muscle stimulation is useful, in maintaining and development of muscle tissue. Carbon based rubber and sponges are the typical electrodes used in nerve and muscle stimulation, providing a uniform electrical coupling across the skin electrode interface

The conductive surface is the layer that faces toward the subject's skin when the electrode is installed on the subject. The electrode pad allows for larger area of distribution of the muscle stimulation. Electrode pads suitable for nerve and/or muscle stimulation may be relatively large having dimensions of several inches or more. Current carbon electrode pads are interchanged between patients. They are usually cleaned after each patient with an antimicrobial solution. A gel or some other electrolyte solution is then applied to the pad prior to reapplication on the next patient's skin.

The industry has not yet met the need of a disposable covering utilized with existing electrodes that will permit transmission of electric pulse to the patient's skin yet provide a protective barrier that is disposable when electrodes are used on subsequent patients.

### SUMMARY OF THE INVENTION

The present invention provides pre moistened disposable cover, also known as pads, that contains a electrolyte solution, eliminating the need for electrolyte gels or other solutions. These buffer covers provide a hygienic protective layer between the patient and the electrode pads, therefore reducing risk of cross contamination of skin conditions.

In one embodiment, the invention electrode cover for use with a medical electrode, said cover comprising:

a main body having a substantially planer configuration, said main body having a distal surface constructed to mate with an electrode and a proximal surface constructed for placement on a user's skin, said main body constructed and arranged with thickness and porosity to deliver 95.0-99.9% of electrical energy transmitted from said medical electrode to the skin surface of a patient.

The cover is constructed and arranged to mate with at least one of an Electrical Muscle Stimulation (E.M.S.) electrode or Transcutaneous Electrical Nerve Stimulation (T.E.N.S.) electrode.

The cover is constructed and arranged to be electrically conductive.

The cover is constructed and arranged to be provided to a user, pre-absorbed with electrolyte solution embedded therein.

The cover is constructed and arranged to be provided to a user, pre-absorbed with electrolyte solution and a medicament embedded therein.

The cover is constructed and arranged to contribute to and facilitate iontophoretic delivery of a medicament.

Also contemplated is a system including the cover pads with a solution to facilitate therapy.

### BRIEF DESCRIPTION OF THE SEVERAL VIEWS OF THE DRAWINGS

FIG. 1 is a view of the components used in the present invention.

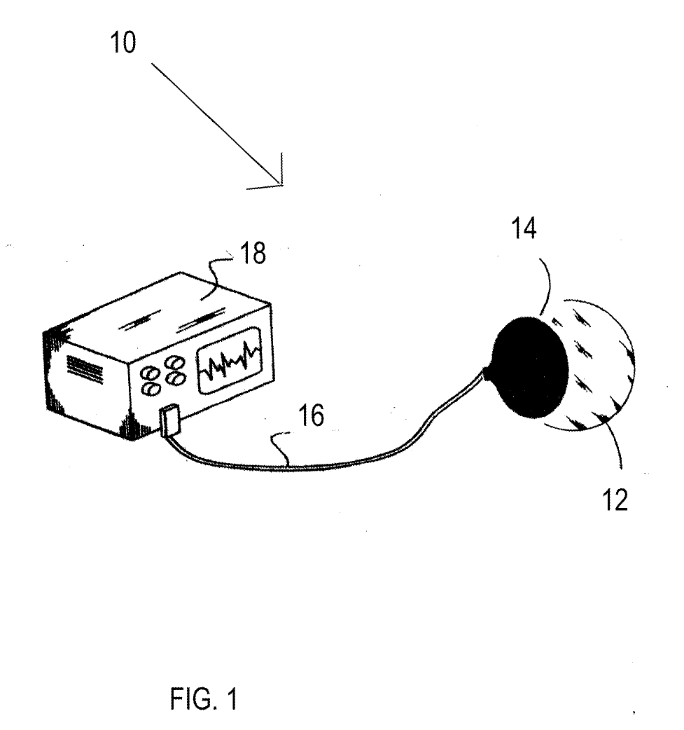

FIG. 2 is a plan view of one cover of the present invention.

### DETAILED DESCRIPTION OF THE PREFERRED EMBODIMENT

The invention is intended for use in medical applications, specifically as it relates to the electrodes used in Electrical Muscle Stimulation (E.M.S.) and Transcutaneous Electrical Nerve Stimulation (T.E.N.S.) Medical electrodes have in the past taken many shapes and forms. Principally, they have been shaped according to the use with the body part for which they are intended. One objective is for an electrode is to maintain good electrical signals, allowing adequate transmission between a patient's skin surface and the electrical wiring connected to the Electrical Muscle Stimulator. Transmission to the skin also needs to be free of current concentration points or “hot spots.” Local “hot spots” can cause discomfort to the patient, if not causing burns to the patient's skin.

System 10 utilizes electrode cover 12 of the present invention. Cover 12 is constructed and arranged to function with electrode pad 14 that receives electrical energy supplied through transmission line 16 that is connected to electrical power supply 18.

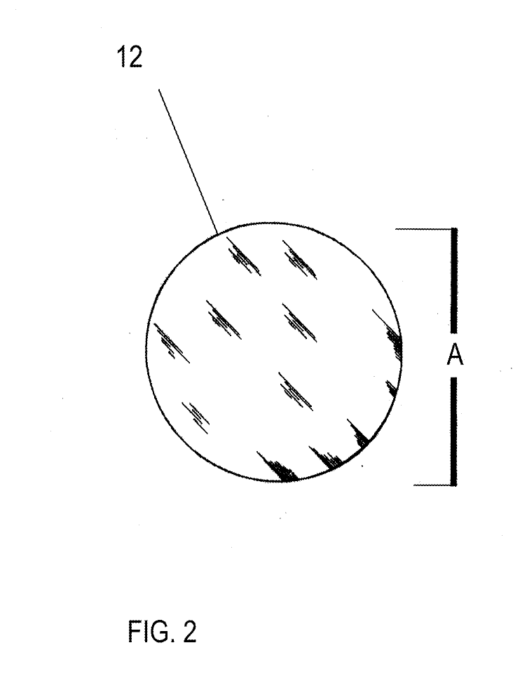

As seen in FIG. 2, cover 12 has a diameter A that is variable as desired. In one embodiment, diameter A is between about 1-4 inches. In another embodiment, A is between 2-4 inches. One preferred embodiment is a diameter of 2.5-3.5 inches.

Although the figure shows a round cover, the geometric configuration is variable and includes, rectangles and other symmetrical and asymmetrical shapes that are utilized as needed.

In one embodiment, the shape is congruous with a particular anatomical shape in order to facilitate placement and delivery of electrical impulses to a specific body part.

Some electrodes are made of a carbon based rubber or sponge material that contact the patients skin. The layer that touches the skin transmits an electrical signal to the patient's skin and muscles. The electrode are flexible enough to mold to the shape of the body part being treated as well as increase the surface area of the electrical impulse. Carbon and sponge electrodes require that the surface touching the skin be coated with an electrode gel, paste or other electrolyte solution. These solutions serve to enhance the conductivity across the skin-pad interface and helps maintain a constant, efficient and effective electrical transmission for longer periods of time.

One objective of this invention, therefore, is to provide a protective buffer pad between the interface of the electrode and the patient's skin that will eliminate the need for an electrolyte solution, electrode paste or electrode gel. Because an electrode suitable for nerve and/or muscle stimulation must provide for an electrical signal to be distributed over the entire surface of the electrode, the buffer pad contacting the electrode must be conductive.

Another objective, therefore, is to maintain the conductivity of the electrode, wherein the buffer pad composition is electrically conductive, with the conductivity being uniform throughout the pad. This will be achieved by having the buffer pads pre-absorbed with electrolyte solution.

A further objective is that the buffer pad is hypoallergenic, and provides a hygienic barrier between the electrode and the patient's skin. It has been found that the use of reusable electrodes in nerve and/or muscle stimulation results in a skin rash in up to 25% to 35% of the people undergoing treatment. Because the electrode is separated by the buffer pad and never touches the patients skin, the risk of cross contamination of skin conditions such as scabies, ring worm and even MRSA is greatly reduced.

Additional objective is to help maintain proper electrode positioning. Current carbon electrodes require a gel or other electrolyte solution to achieve proper electrical coupling. Nerve and/or muscle stimulation causes muscle contraction, resulting in a considerable amount of skin movement. The composition of the carbon rubber pads combined with the gels, results in frequent slippage. A physician, or the like of course, performs precision positioning of the electrode, with knowledge in the treatment method. Inaccurate placement of the electrode, or slipping of the electrode from its intended position, may significantly reduce the beneficial effects of the treatment. The textures of the buffer pads provide adequate friction between the electrode and the patient's skin, to prevent slippage during treatment. The nature of the fabric allows it to conform to the shape of the electrode and patient's skin, to maintain continuous placement. This helps prevent patient discomfort due to “hot spots”.

Further objective is to have protective buffer pads offered in multiple forms. One form is a natural, hypoallergenic pad that is safe for people with sensitive skin, with no additives and/or dyes. The second pad will be combined with skin safe topical analgesics such as Biofreeze™” or Aspercreme™. The third pad will be available for prescription based pain medications such as “Fentynal”. Used in conjunction with the Electrical Muscle Stimulation, the present invention provides an additional benefit of Iontopheresis.

Iontopheresis is a type of physical process in which ions flow diffusively in a medium, driven by an electric field. The analgesic solution in the buffer pad is driven by the electrical current of the electrode pad into the skin, and is pushed deeper into the skin tissue.

Another objective is to prolong the longevity of the carbon pads that are currently being used for Electrical Muscle Stimulation. Current pads require cleaning with harsh chemicals between patient use. These chemicals break down the carbon rubber material of the electrode pads requiring frequent replacement. These chemicals also put patients at a greater risk due to irritation from the chemicals in the cleaning solution. The buffer pads are hypoallergenic and eliminate the need for harsh cleaning solutions.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. An electrode cover for use with a medical electrode, said cover comprising:
a main body having a substantially planer configuration, said main body having a distal surface constructed to mate with an electrode and a proximal surface constructed for placement on a user's skin, said main body constructed and arranged with thickness and porosity to deliver 95.0-99.9% of electrical energy transmitted from said medical electrode to the skin surface of a patient.

2. The cover of claim 1 constructed and arranged to mate with at least one of an Electrical Muscle Stimulation (E.M.S.) electrode or Transcutaneous Electrical Nerve Stimulation (T.E.N.S.) electrode.

3. The cover of claim 1 constructed and arranged to be electrically conductive.

4. The cover of claim 1 constructed and arranged to be provided to a user, pre-absorbed with electrolyte solution embedded therein.

5. The cover of claim 1 The cover of claim 1 constructed and arranged to be provided to a user, pre-absorbed with electrolyte solution and a medicament embedded therein.

6. The cover of claim 1 constructed and arranged to contribute to and facilitate iontophoretic delivery of a medicament.
